(19) United States
(12) Patent Application Publication
(10) Pub. No.: US 2013/0252945 A1
(43) Pub. Date: Sep. 26, 2013

(54) AMORPHOUS BIOINORGANIC IONIC LIQUID COMPOSITIONS COMPRISING PHARMACEUTICALS

(71) Applicant: LOS ALAMOS NATIONAL SECURITY, LLC, Los Alamos, NM (US)

(72) Inventors: Katherine Lovejoy, Santa Fe, NM (US); Rico Del Sesto, St. George, UT (US); Andrew Koppisch, Flagstaff, AZ (US)

(21) Appl. No.: 13/849,993

(22) Filed: Mar. 25, 2013

Related U.S. Application Data
(60) Provisional application No. 61/685,767, filed on Mar. 23, 2012.
(60) Provisional application No. 61/638,605, filed on Apr. 26, 2012.

(51) Int. Cl. A61K 47/02

(52) U.S. Cl. CPC A61K 47/02
USPC 514/217; 514/643; 514/304; 514/556; 514/471; 514/619; 514/642; 514/356

(57) ABSTRACT

An amorphous formulations of a pharmaceutical includes a composition of the formula [A]ₓ[MₚCl_{q}]_{y}, the composition being an ionic liquid with a melting temperature below 150° C., wherein [MₚCl_{q}] is a metal chloride, x is 1, 2, 3, or 4, p is 1, 2, 3, or 4, q is 1, 2, 3, 4, 5, 6, 7, 8, or 9, and y is 1 or 2, wherein each A is a cation that is a pharmaceutical or a cation precursor that is a pharmaceutical.

### RELATED APPLICATIONS

This application claims the benefit of U.S. Provisional Patent Application No. 61/685,767 entitled “Bioinorganic Ionic Liquid Formulations of Pharmaceuticals,” filed Mar. 23, 2012, and U.S. Provisional Patent Application No. 61/638,605 entitled “Bioinorganic Ionic Liquid Formulations of Pharmaceuticals,” filed Apr. 26, 2012, both hereby incorporated by reference.

### STATEMENT REGARDING FEDERAL RIGHTS

This invention was made with government support under Contract No. DE-AC52-06NA25396 awarded by the U.S. Department of Energy. The government has certain rights in the invention.

### FIELD OF THE INVENTION

The present invention relates generally to ionic liquid (IL) compositions and more particularly to amorphous, thermally stable metal chloride-based IL compositions, to the synthesis of these metal chloride-based IL compositions, and to the use of these metal chloride-based IL compositions.

### BACKGROUND OF THE INVENTION

Stable, bioavailable, amorphous formulations of pharmaceuticals are needed for modern medicine. Amorphous formulations of pharmaceuticals are often more soluble than crystalline formulations (e.g. salts) of pharmaceuticals. In addition, bioavailability of amorphous pharmaceuticals is not affected by crystal polymorphism (for more detail, see: Babu, N. Jagadeesh et al., Cryst. Growth Des. 2011, 11:2662-2679).

Current strategies for production of amorphous pharmaceuticals include rapid cooling, lyophilization, and production of ionic liquids from organic ions. These strategies do not predictably form amorphous products. Instead, these strategies result in products that can crystallize over time, or they result in products that are less stable than crystalline formulations are (for more detail, see: Hancock, B. C. et al., J. Pharm. Sci. 1997, 86:1-12).

Initial applications of organic-only ionic liquids to biological systems have included stabilization of proteins (see: Baker, S. N. et al., Chem. Commun. 2004, 940-941) and pharmaceuticals (see: Mizuuchi, H. et al., Eur J Pharm Sci 2008, 33:326-331) in solution, and demonstration of antibiofilm (see: Busetti, Alessandro et al., Green Chem. 2010, 12:420-425) and antifungal (see: Davis, James H., Jr. et al., Tetrahedron Lett. 1998, 39:8955-8958) activity. They have also been used as crystallization solvents for designing pharmaceutical polymorphs, as for adefovir dipivoxil (as described in: An, Ji-Hun et al., Cryst. Growth Des. 2010, 10:3044-3050). Ionic liquid drug pharmaceutical formulations pair cations and anions, at least one of which is pharmaceutically active, to produce an amorphous molten salt that melts near room temperature and generally improves thermal stability, solubility, release rates, bioavailability, and ease of use and manufacture, and also circumvents issues related to crystal polymorphism (for more detail, see: Hough, Whitney L. et al., New J. Chem. 2007, 31:1429-1436). The use of GRAS substances can reduce regulatory roadblocks to new pharmaceutical formulations. Known IL formulations comprising all-GRAS components include rantidinium docusate, benzalkonium ibuprofate (for more detail, see: Hough, Whitney L. et al., New J. Chem. 2007, 31:1429-1436) and saccharinate, (see: Stoimenovski, J. et al., Pharm. Res. 2010, 27:521-526) and procainamidium and lidocanium salicylate (for more detail, see: Bica, K. et al., Chem. Commun. 2010, 46:1215-1217).

Although amorphous ionic liquid formulations have been found for some pharmaceuticals, difficulty in predicting which organic cation-anion pairs would form amorphous melts led us to seek what we believe is a more reliable strategy.

A common problem that currently exists with many pharmaceuticals is low solubility. Low solubility can make formulating a particular compound difficult, and generally low solubility translates into low bioavailability. Much research is conducted on finding ways to improve a compound's solubility and availability. Typically methods include complex delivery devices and chemical modifications of the pharmaceutical to a more soluble formulation.

Surfaces designed for use in aquatic, marine, or other aqueous environments require coating them with a film to prevent biofouling by prokaryotes and eukaryotes such as algae, bacteria, or other microorganisms, and barnacles or other macroorganisms. Currently, copper-containing bottom paint is used for watercraft, but it is not transparent. Durable, transparent surface coatings that prevent biofouling of sensors in aqueous environments are desirable.

Amorphous compositions of pharmaceutical and agricultural substances are desirable. Amorphous compositions of substances that are generally-recognized-as-safe (“GRAS”) substances are desirable.

It is also desirable is that the chemical, biological, and physical properties of these amorphous compositions be tunable. It is also desirable that these amorphous compositions be thermally stable and not be subject to polymorphism, and for which controlled, tunable dissolution and solubility are possible.

Methods of preparing and using these amorphous compositions are desirable. Methods of converting a compound that is difficult to solubilize into a more soluble formulation are desirable.

Methods of converting a thermally unstable compound into a more thermally stable form are also desirable.

### SUMMARY OF THE INVENTION

In accordance with the purposes of the disclosed compositions, methods, and devices, as embodied and broadly described herein, the disclosed subject matter, in one aspect, relates to compositions and methods for preparing and using such compositions. In a further aspect, the disclosed subject matter relates to chlorometallate-based ionic liquid compositions that can be used for or in pharmaceutical applications. The compositions are amorphous. The amorphous nature is forced by the presence of multiple chlorometallate species. The compositions include those of the formula [A]ₓ[MₚCl_{q}]_{y}, said composition being an ionic liquid with a melting temperature below about 150° C., wherein [MₚCl_{q}] is a metal chloride, x is 1, 2, 3, or 4, p is 1, 2, 3, or 4, q is 1, 2, 3, 4, 5, 6, 7, 8, or 9, and y is 1 or 2, wherein each A is a cation that is a pharmaceutical substance, or wherein each A is a cation precursor that is a substance.

Another aspect of the present invention relates to methods for making the disclosed ionic liquid compositions. Another aspect relates to using the compositions. Another aspect relates to overcoming problems related to low solubility, low thermal stability, release rate, and biofouling problems, and to improve ease of use and manufacture.

Yet another aspect of the invention relates to a composition of the formula [A]ₓ[MₚCl_{q}]_{y}, wherein [MₚCl_{q}] is selected from Zn₄Cl₁₀, ZnCl₄, Zn₃Cl₈, Zn₂Cl₆, ZnCl₃, Zn₂Cl₅, or Zn₃Cl₇ and A is a cation or cation precursor selected from benzethonium, ranitidinium, phenoxybenzamine, homatropine, lysine, procainamide, ethambutol, nicardipine, or imipramine or a cationic derivative thereof.

Another aspect of the invention relates to a method for preventing biofouling on surfaces or sensors immersed in aquatic environments using a thin layer of clear, colorless ionic liquid of the formula [A]ₓ[MₚCl_{q}]_{y}, wherein [MₚCl_{q}] is a metal chloride, x is 1, 2, 3, or 4, p is 1, 2, 3, or 4, q is 1, 2, 3, 4, 5, 6, 7, 8, or 9, and y is 1 or 2.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### DETAILED DESCRIPTION

In an aspect of the invention, a broadly applicable, anti-crystal engineering approach (for more detail, see: Dean, Pamela M. et al., Cryst. Growth Des. 2009, 9:1137-1145) was demonstrated for synthesizing thermally stable, amorphous formulations of cationic pharmaceuticals using generally-recognized-as-safe (GRAS) metal chlorides. The strategy, which is known for producing amorphous ionic liquids for other applications, is for the first time applied for amorphous pharmaceutical formulations. The approach is generally applicable to any pharmaceutical that can be synthesized as a chloride salt with a melting point lower than about 300° C., and has produced an agent with potent anti-biofilm activity.

An aspect of this invention lies in the use of GRAS metal chlorides to reliably and predictably produce highly stable amorphous formulations of pharmaceutically relevant chloride salts. It should be noted that previous efforts by others have developed the science of amorphous chlorometallate melts, shown that metals are acceptable for use in medicine, examined potential interactions of ionic liquids and biological systems, and unveiled the advantages and disadvantages of applying all-organic ionic liquids for pharmaceutical purposes.

An aspect of this invention relates to the synthesis of chlorometallate ionic liquids (ILs) that are ionic liquid formulations of known pharmaceuticals. Until now, chlorometallate ILs are unexplored for the purpose of pharmaceutical applications.

Chlorometallate ILs are formed by combining a metal chloride and an organic chloride salt, with Lewis acidity or basicity dependent on the MClₓ: organic chloride salt ratio (for more detail, see: Melton, T. J. et al., J. Electrochem. Soc. 1990, 137:3865-3869). A 2:1 ratio introduces excess chloride and forces an amorphous melt by formation of multiple fluidizing chlorometallate species (for more detail, see: Wilkes, John S. et al., Inorg. Chem. 1983, 22:3870-3872).

Although unexplored for the purpose of pharmaceutical applications, chlorometallate ILs are known for other applications. For example, the chloroaluminates have found use as electrolytes, catalysts, and solvents (for more detail, see: Wilkes, J. S. et al., Inorg. Chem. 1982, 21:1263-1264). Chlorometallates that form ILs include ZnCl₂, SnCl₂, FeCl₃, InCl₃, GaCl₃, NiCl₃, CoCl₂, MnCl₂, and GdCl₃ (for more detail, see: Abbott, A. P. et al., Inorg. Chem. 2004, 43:3447-3452).

Several of the species that form amorphous ionic liquids due to chlorometallate speciation are also suitable for pharmacological use. The common compound zinc(II) chloride, for example, is found in food and medicine and is on the FDA's list of Generally Recognized as Safe substances (1973, 21 CFR Section 182.8985) and, when combined with chloride salts of organic molecules, forms amorphous gels that melt at temperatures of from −30-100° C. (for more detail, see: Abbott, A. P. et al., Chem. Commun. 2001, 2010-2011) Zinc salts and complexes are found in filet mignon (10 mg/serving) and even in doubly distilled H₂O (“ddH₂O”), wherein upwards of 1 micromolar (“μM”) zinc persists due to trace zinc in plastic tubing. Besides ZnCl₂, many other metals are used in FDA-approved medicines including, for example, iron(III), platinum(II), bismuth(III), Ag(I), and Au(I). Beyond metals that contribute as active agents in FDA-approved medicines, an even larger portion of the periodic table is allowable in formulations and classified as GRAS.

The materials, compounds, compositions, articles, and methods described herein may be understood more readily by reference to the following detailed description of specific aspects of the disclosed subject matter and the Examples included therein.

Before the present compositions, methods, articles, and devices are disclosed and described any further, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### GENERAL DEFINITIONS

In this specification and in the claims that follow, reference will be made to a number of terms, which shall be defined to have the following meanings:

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an ionic liquid” includes mixtures of two or more such ionic liquids, reference to “the compound” includes mixtures of two or more such compounds, and the like.

“Optional” or “optionally” means that the subsequently described event or circumstance can or cannot occur, and that the description includes instances where the event or circumstance occurs and instances where it does not.

### CHEMICAL DEFINITIONS

The term “ion,” as used herein, refers to any molecule, portion of a molecule, cluster of molecules, molecular complex, moiety, or atom that contains a charge (positive, negative, or both (e.g., zwitterions)) or that can be made to contain a charge. Methods for producing a charge in a molecule, portion of a molecule, cluster of molecules, molecular complex, moiety, or atom are disclosed herein and can be accomplished by methods known in the art, e.g., protonation, deprotonation, oxidation, reduction, alkylation, etc.

The term “anion” is a type of ion and is included within the meaning of the term “ion.” An “anion” is any molecule, portion of a molecule (e.g., Zwitterion), cluster of molecules, molecular complex, moiety, or atom that contains a net negative charge or that can be made to contain a net negative charge. The term “anion precursor” is used herein to specifically refer to a molecule that can be converted to an anion via a chemical reaction (e.g., deprotonation).

The term “cation” is a type of ion and is included within the meaning of the term “ion.” A “cation” is any molecule, portion of a molecule (e.g., Zwitterion), cluster of molecules, molecular complex, moiety, or atom, that contains a net positive charge or that can be made to contain a net positive charge. The term “cation precursor” is used herein to specifically refer to a molecule that can be converted to a cation via a chemical reaction (e.g., protonation or alkylation).

The term “bioactive property” is any local or systemic biological, physiological, or therapeutic effect in a biological system. For example, the bioactive property can be the control of infection or inflammation, enhancement or suppression of growth, action as an analgesic, anti-viral, pesticidal, herbicidal, or nutrientional action, etc. Many examples of bioactive properties are disclosed herein.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

Reference will now be made in detail to specific aspects of the disclosed materials, compounds, compositions, articles, and methods, examples of which are illustrated in the accompanying Examples.

### Materials and Compositions

In one aspect, disclosed herein are ionic liquid compositions. The term “ionic liquid” has many definitions in the art, but is used herein to refer to salts (i.e., compositions comprising cations and anions) that are liquid at a temperature of at or below about 150° C. That is, at one or more temperature ranges or points at or below about 150° C., the disclosed ionic liquid compositions are liquid; although, it is understood that they can be solids at other temperature ranges or points. Since the disclosed ionic liquid compositions are liquid, and thus not crystalline solids, at a given temperature, the disclosed compositions do not suffer from the problems of polymorphism associated with crystalline solids.

The use of the term “liquid” to describe the disclosed ionic liquid compositions is meant to describe a generally amorphous, non-crystalline, or semi-crystalline state. For example, while some structured association and packing of cations and anions can occur at the atomic level, the disclosed ionic liquid compositions have minor amounts of such ordered structures and are therefore not crystalline solids. The compositions disclosed herein can be fluid and free-flowing liquids or amorphous solids such as glasses or waxes at a temperature at or below about 150° C. In particular examples disclosed herein, the disclosed ionic liquid compositions are liquid at the body temperature of a subject.

It is further understood that the disclosed ionic liquid compositions can include solvent molecules (e.g., water); however, these solvent molecules should not be present in excess in the sense that the disclosed ionic liquid compositions are dissolved in the solvent, forming a solution. That is, the disclosed ionic liquid compositions contain no or minimal amounts of solvent molecules that are free and not bound or associated with the ions present in the ionic liquid composition. Thus, the disclosed ionic liquid compositions can be liquid hydrates or solvates, but not solutions.

The ionic liquid compositions disclosed herein are comprised of at least one kind of anion and at least one kind of cation. The at least one kind of cation can be a pharmaceutical active, a pesticidal active, a herbicidal active, a food additive, a nutraceutical, or the like, including any combination thereof, as is disclosed herein. It is contemplated that the disclosed ionic liquid compositions can comprise one kind of cation with more than one kind of anion (e.g., 2, 3, 4, 5, 6, 7, 8, 9, 10, or more different kinds of anions). Likewise, it is contemplated that the disclosed ionic liquid compositions can comprise one kind of anion with more than one kind of cation (e.g., 2, 3, 4, 5, 6, 7, 8, 9, 10, or more different kinds of cations). Further, the disclosed ionic liquids can comprise more than one kind of anion (e.g., 2, 3, 4, 5, 6, 7, 8, 9, 10, or more different kinds of anions) with more than one kind of cation (e.g., 2, 3, 4, 5, 6, 7, 8, 9, 10 or more different kinds of cations).

In addition to the cations and anions, the ionic liquid compositions disclosed herein can also contain nonionic species, such as solvents, preservatives, dyes, colorants, thickeners, surfactants, viscosity modifiers, mixtures and combinations thereof and the like. However, the amount of such nonionic species is typically low (e.g., less than about 10, 9, 8, 7, 6, 5, 4, 3, 2, or 1 wt. % based on the total weight of the composition). In some examples described herein, the disclosed ionic liquid compositions are neat; that is, the only materials present in the disclosed ionic liquids are the cations and anions that make up the ionic liquid compositions. It is understood, however, that with neat compositions, some additional materials or impurities can sometimes be present, albeit at low to trace amounts (e.g., less than about 10, 9, 8, 7, 6, 5, 4, 3, 2, or 1 wt. % based on the total weight of the composition).

The disclosed ionic liquid compositions are liquid at some temperature range or point at or below about 150° C.

It is understood that the disclosed ionic liquid compositions can, though need not, be solubilized, and solutions of the disclosed ionic liquids are contemplated herein. Further, the disclosed ionic liquid compositions can be formulated in an extended or controlled release vehicle, for example, by encapsulating the ionic liquids in microspheres or microcapsules using methods known in the art. Still further, the disclosed ionic liquid compositions can themselves be solvents for other solutes. For example, the disclosed ionic liquids can be used to dissolve a particular nonionic or ionic pharmaceutical active. These and other formulations of the disclosed ionic liquids are disclosed elsewhere herein.

In some examples, the disclosed ionic liquids are not solutions where ions are dissolved in a solute. In other examples, the disclosed ionic liquid compositions do not contain ionic exchange resins. In still other examples, the disclosed ionic liquids are substantially free of water. By substantially free is meant that water is present at less than about 10, 9, 8, 7, 6, 5, 4, 3, 2, 1, 0.5, 0.25, or 0.1 wt. %, based on the total weight of the composition.

In some particular examples, one or more ions in the disclosed ionic liquid composition (e.g., the anions, cations, or both) can be a pharmaceutical active, e.g., an existing drug that is ionic or that can be made ionic. Many drugs exist naturally or at physiological conditions as an ion, or they can be converted to ions via simple chemical transformations (e.g., alkylation, protonation, deprotonation, etc.). As such, these drugs can be used to prepare an ionic liquid composition as disclosed herein. Such drugs can possess any therapeutic or prophylactic activity, many of which are described herein. Combining such drugs with other ions to prepare an ionic liquid, as is disclosed herein, can result in the modification and/or enhancement of the drug's properties. For example, a first drug ion with a given property can be combined with an oppositely charged second ion with another property to effect the controlled release, controlled delivery, biological impact, taste, physical properties (stability, solubility, toxicity, melting point, etc.), or to overcome polymorphism in the first drug ion. In this way, new drug compositions can be created by forming ionic liquids with functionality crafted into the combination of the ions, as disclosed herein.

Because the disclosed ionic liquid compositions can have multiple functionalities or properties, each arising from the various ions that make up the ionic liquid, the disclosed ionic liquid compositions can be custom designed for numerous uses. As disclosed herein, any combination of cations and anions, as disclosed herein, can be made as long as the combination results in an ionic liquid as described herein. That is, any compound or active disclosed herein that has a given charge or can be made to have a given charge (the “first ion(s)”) can be combined with any other compound or active disclosed herein having a charge opposite to that of the first ion(s) or any compound that can be made to have a charge opposite to that of the first ion(s). Thus, in many examples, the ionic liquid compositions can have one type of cation and one type of anion, in a 1:1 relationship, so that the net charge of the ionic liquid is zero.

Furthermore, many of the ions disclosed herein can have multiple charges. Thus, when one ion having a multiple charge is used, more counterion(s) is needed, which will affect the ratio of the two ions. For example, if a cation having a plus 2 charge is used, then twice as much anion having a minus 1 charge is needed. If a cation having a plus 3 charge is used, then three times as much anion having a minus 1 charge is needed, and so on. While the particular ratio of ions will depend on the type of ion and their respective charges, the disclosed ionic liquids can have a cation to anion ratio of 1:1:, 2:1, 3:1, 4:1, 1:3, 2:1, 3:2, 2:3, and the like.

### Methods

Further, when preparing an ionic liquid composition as disclosed herein, molecular asymmetry can be particularly desired. Low-symmetry cations and anions typically reduce packing efficiency in the crystalline state and lower melting points.

Many of the bioactive compounds (e.g., pharmaceutical actives, pesticidal actives, herbicidal actives, etc.) and energetic compounds disclosed herein are cationic or can be made cationic, the identification of which can be made by simple inspection of the chemical structure as disclosed herein. Further, many of these compounds are commercially available as their halide salts or can be converted to their halide salts by ion exchange chromatography or reactions with acids (e.g. HCl, HBr, or HI).

### Uses

The disclosed ionic liquid compositions have many uses. For example, the disclosed ionic liquid compositions can be used to allow fine tuning and control of the rate of dissolution, solubility, and bioavailability, to allow control over physical properties and mechanical strength, to improve homogenous dosing, and to allow easier formulations. The disclosed ionic liquid compositions also make having compositions with additional functionality possible.

Generally, any use that exists for one or more of the ionic components in the ionic liquid is also a use for the ionic liquid composition itself. For example, if one of the ions in an ionic liquid composition disclosed herein is a pharmaceutical active, then the ionic liquid composition can also be used for the same indication as the pharmaceutical active.

Some pharmaceutical actives that are envisioned as ionic components of embodiment ionic liquid compositions include, but are not limited to, aspirin, chlordiazepoxide HCl, isoniazid, penicillin, sulfamidochrysoidine, cisplatin, 6-mercaptopurine, chimeric anti-CD20 antibody, paclitaxel, phenobarbital, fluoxetine, fexofenadine, rofecoxib, quinine, ivermectin, L-dopa, 10-(3-dimethylaminoproply)-2-chlorphenothiazine, salvarsan, cimetidine, zidovudine, indinavir sulfate, salbutamol, digoxin, fluride, zidovudine, erythropoietin, hydrocortisone, insulin, oral contraceptive, oxytocin, mifepristone, thyroxine, thalidomide, cyclosporine, fentanyl, methadone, morphine, botox, vitamins, methylphenidate, sildenafil, pantoprazole, rabeprazole, risedronate, alendronate, losartan, fosinopril, atorvastatin, pravastatin, montelukast, 2-aminothiazole, 6-azauridine, 6-mercaptopurine, 9-aminocamptothecin, acetarsone, acetazolamide, acetosulfone, acetylcysteine, acetylpheneturide, acranil, acrivastine, adrenalone, albutoin, aldioxa, alibendol, alizapride, alkofanone, allopurinol, ambenonium, ambroxol, ambutonium, amexinium, aminitrozole, amiphenazole, amisulpride, anagrelide, arsanilate, arshpenamine, asoxime, azidamfenicol, benfluorex, benfunolol, benserazide, benzaepril, benzilonium, benznidazole, benzoxonium, benzoylpas, berberine, bibenzonium, brequinar, brodimoprim, bromhexine, bromopride, bromo-salicylchloranilide, bucillamine, buformin, bufuralol, bumadizon, bunitrolol, buthalital, cabergoline, carbachol, carbarsone, carbidopa, cariporide, carnitine, caroxazone, celecoxib, cephaeline, cetalkonium, chloguanide, chloramphenicol, chlordantoin, chlorisonclamine, chloroquine, cholic acid, choline, ciclonicate, cilostazol, cimetidine, cinepazide, cinitapride, cisapride, clanobutin, clometocillin, clomiphene, cloricromen, clortermine, cyamelide, cyclazocine, dapsone, dimefline, diphetarsone, distigmine, dithiazanine, donepezil, dopamine, echothiophate, edrophonium, efavirenz, elformithine, enfenamic acid, etifoxine, etodroxizine, fampridine, fazadinium, fendiline, fenproporex, finasteride, flufenamic acid, flumethiazide, fluoxetine, flurazepam, flutamide, fomepizole, fominoben, hexafluorenium, hydroxystilbamidine, ibudilast, indalpine, indeloxazine, ipratropium, irsogladine, isobutyl p-aminobenzoate, isoetharine, levosimendan, lisuride, lodoxamide, mesalamine, mesoxalate, metformin, methionine, methoxyphenamine, methyl hexaneamine, methylene blue, methylthiouracil, metrons, metyrosine, minoxidil, nadoxolol, nafronyl, naphazoline, naratriptan, neostigmine, nicametate, niclosamide, nicorandil, nilutamide, niridazole, nitazoxanide, nordefrin, obidoxime, omapatrilat, orotic acid, oxaceprol, oxitropium, ozagrel, pamidronic acid, p-aminobenzoic acid, p-aminopropiophenon, pelletierine, pemoline, penicillamine, pentamethonium, pentamidine, pentoxifylline, pergolid, perhexiline, phenmetrazine, phentolamine, phenylephrine, picotamide, pidotimod, pimobendan, pinacidil, pramipexole, prenylamine, prinomastat, pyridinol carbamate, pyridoxine, pyrimethamine, ramatroban, remoxipride, repinotan, riluzole, ritodrine, succinimide, succinylcholine, sulfamethoxazole, sulfapyridine, sulpiride, synephrine, tamsulosin, tedisamil, tenuazonic acid, terazosin, terbutaline, tetrazepam, tetroxoprim, thiabendazole, thiamine, thiopental, tiapride, timonacid, tiopronin, tiotropium, tizanidine, tolazoline, tropicamide, valnoctamide, zafirlukast or a cationic derivative thereof.

Depending on the particular ions, the disclosed ionic liquid compositions can be used to treat a subject diagnosed with, for example, endocrine disorders, diabetes, infertility, hormone deficiencies, osteoporosis, ophthalmological disorders, neurodegenerative disorders, Alzheimer's disease, dementia, Parkinson's disease, multiple sclerosis, Huntington's disease, cardiovascular disorders, atherosclerosis, hyper-coagulable states, hypo-coagulable states, coronary disease, cerebrovascular events, metabolic disorders, obesity, vitamin deficiencies, renal disorders, renal failure, haematological disorders, anemia of different entities, immunologic and rheumatologic disorders, autoimmune diseases, immune deficiencies, infectious diseases, viral infections, bacterial infections, fungal infections, parasitic infections, neoplastic diseases, multi-factorial disorders, impotence, chronic pain, depression, and different fibrosis states.

Similarly, with herbicidal and pesticidal actives, the ionic liquid compositions disclosed herein that contain ionic pesticidal and herbicidal actives can be used in the same way as the actives themselves. Thus, any use contemplated for a pesticidal and herbicidal active is contemplated herein for an ionic liquid composition containing that active.

Examples of herbicidal actives that are envisioned as ionic components of embodiment ionic liquid compositions include, but are not limited to metribuzin, benefin, ethoxysulfuron, flumetsulam, metosulam, nicosulfuron, prosulfuron, rimsulfuron, thifensulfuron-methyl, ametryn, mepiquat, mepiquat chloride, amitrole, piperazine, butylamine, haloxydine, pyriclor, chlormequat, choline, aviglycine, tiaojiean, clopyralid-methyl, chlorthiamid, eglinazine-ethyl, iprymidam, simazine, chloramben-methyl, dichlormid, atrazine, bromoxynil, cyanazine, hexazinone, terbuthylazine, diflufenzopyr, EPTC, fenoxaprop-P-ethyl, glyphosate, pendimethalin, trifluralin, asulam, triaziflam, diflufenican, fluoroxypyr, diflufenzopyr or a cationic derivative thereof.

Examples of pesticidal actives that are envisioned as ionic components of embodiment ionic liquid compositions include, but are not limited to methamidophos, 4-aminopyridine, thiocyclam, clothianidin, cyromazine, benclothiaz, imidaclothiz, dinotefuran, cartap, cartap hydrochloride, carfentrazone-ethyl, sulfentrazone, clomazone, diclofop-methyl, oxamyl propargite, prosulfuron, pyridate, pyriftalid, S-metolachlor, simazine, terbuthylazine, terbutryn, triasulfuron, trifloxysulfuron, trinexapac-ethyl, ametryn, atrazine, benoxacor, bifenthrin, butafenacil, chlortoluron, cinosulfuron, clodinafop, cloquintocet, desmetryn, dicamba, dimethachlor, dimethametryn, fenclorim, flumetralin, fluometuron, fluthiacetmethyl, halosulfuron, isoproturon, metobromuron, metolachlor, norflurazon, oxasulfuron, piperophos, pretilachlor, primisulfuron, prometryn, propaquizafop, acibenzolar-5-methyl, chlorothalonil, cyproconazole, cyprodinil, difenoconazole, fenpropidin, fenpropimorph, furalaxyl, metalaxyl, metalaxyl-M, oxadixyl, penconazole, propiconazole, pyrifenox, thiabendazol, abamectin, bromopropylate, cypermethrin, cypermethrin high-cis, cyromazine, diafenthiuron, diazinon, dichlorvos, disulfoton, emamectinbenzoate, fenoxycarb, formothion, furathiocarb, lufenuron, methidathion, permethrine, codlemone, phosphamidon, profenofos, pymetrozine, quinalphos, terrazole, thiamethoxam, thiocyclam, thiometon, triallate, trifloxystrobin, vinclozolin, zetacypermethrin, prohexadione, or a cationic derivative thereof.

Many of the disclosed ionic liquid compositions can be used therapeutically as neat ionic liquids. Also, the disclosed ionic liquids can be used in combination with a pharmaceutically acceptable carrier. By “pharmaceutically acceptable” is meant a material that is not biologically or otherwise undesirable, i.e., the material may be administered to a subject without causing any undesirable biological effects or interacting in a deleterious manner with any of the other components of the pharmaceutical formulation in which it is contained. The carrier would naturally be selected to minimize any degradation of the active ingredient and to minimize any adverse side effects in the subject, as would be well known to one of skill in the art. In another aspect, many of the disclosed ionic liquids can be used prophylactically, i.e., as a preventative agent, either neat or with a pharmaceutically acceptable carrier. The ionic liquid compositions disclosed herein can be conveniently formulated into pharmaceutical compositions composed of neat ionic liquid or in association with a pharmaceutically acceptable carrier. See e.g., Remington's Pharmaceutical Sciences, latest edition, by E. W. Martin Mack Pub. Co., Easton, Pa., which discloses typical carriers and conventional methods of preparing pharmaceutical compositions that can be used in conjunction with the preparation of formulations of the compounds described herein and which is incorporated by reference herein. Such pharmaceutical carriers, most typically, would be standard carriers for administration of compositions to humans and non-humans, including solutions such as sterile water, saline, and buffered solutions at physiological pH. Other compounds can be administered according to standard procedures used by those skilled in the art. For example, pharmaceutical compositions can also include one or more additional active ingredients such as antimicrobial agents, anti-inflammatory agents, anesthetics, and the like.

Examples of antibacterial agents that are envisioned as ionic components of embodiment ionic liquid compositions include, but are not limited to acedapsone, fosmidomycin, acetosulfone sodium, alamecin, alexidine, amdinocillin, amdinocillin pivoxil, amicycline, amifloxacin, amifloxacin mesylate, amikacin, amikacin sulfate, aminosalicylic acid, aminosalicylate sodium, amoxicillin, amphomycin, ampicillin, ampicillin sodium, apalcillin sodium, apramycin, aspartocin, astromicin sulfate, avilamycin, avoparcin, azithromycin, azlocillin, azlocillin sodium, bacampicillin hydrochloride, bacitracin, bacitracin methylene disalicylate, bacitracin zinc, bambermycins, benzoylpas calcium, berythromycin, betamicin sulfate, biapenem, biniramycin, biphenamine hydrochloride, bispyrithione magsulfex, butikacin, butirosin sulfate, capreomycin sulfate, carbadox, carbenicillin disodium, carbenicillin indanyl sodium, carbenicillin phenyl sodium, carbenicillin potassium, carumonam sodium, cefaclor, cefadroxil, cefamandole, cefamandole nafate, cefamandole sodium, cefaparole, cefatrizine, cefazaflur sodium, cefazolin, cefazolin sodium, cefbuperazone, cefdinir, cefepime, cefepime hydrochloride, cefetecol, cefixime, cefmenoxime hydrochloride, cefmetazole, cefmetazole sodium, cefonicid monosodium, cefonicid sodium, cefoperazone sodium, ceforanide, cefotaxime sodium, cefotetan, cefotetan disodium, cefotiam hydrochloride, cefoxitin, cefoxitin sodium, cefpimizole, cefpimizole sodium, cefpiramide, cefpiramide sodium, cefpirome sulfate, cefpodoxime proxetil, cefprozil, cefroxadine, cefsulodin sodium, ceftazidime, ceftibuten, ceftizoxime sodium, ceftriaxone sodium, cefuroxime, cefuroxime axetil, cefuroxime pivoxetil, cefuroxime sodium, cephacetrile sodium, cephalexin, cephalexin hydrochloride, cephaloglycin, cephaloridine, cephalothin sodium, cephapirin sodium, cephradine, cetocycline hydrochloride, cetophenicol, chloramphenicol, chloramphenicol palmitate, chloramphenicol pantothenate complex, chloramphenicol sodium succinate, chlorhexidine phosphanilate, chloroxylenol, chlortetracycline bisulfate, chlortetracycline hydrochloride, cinoxacin, ciprofloxacin, ciprofloxacin hydrochloride, cirolemycin, clarithromycin, clinafloxacin hydrochloride, clindamycin, clindamycin hydrochloride, clindamycin palmitate hydrochloride, clindamycin phosphate, clofazimine, cloxacillin benzathine, cloxacillin sodium, cloxyquin, colistimethate sodium, colistin sulfate, coumermycin, coumermycin sodium, cyclacillin, cycloserine, dalfopristin, dapsone, daptomycin, demeclocycline, demeclocycline hydrochloride, demecycline, denofungin, diaveridine, dicloxacillin, dicloxacillin sodium, dihydrostreptomycin sulfate, dipyrithione, dirithromycin, doxycycline, doxycycline calcium, doxycycline fosfatex, doxycycline hyclate, droxacin sodium, enoxacin, epicillin, epitetracycline hydrochloride, erythromycin, erythromycin acistrate, erythromycin estolate, erythromycin ethylsuccinate, erythromycin gluceptate, erythromycin lactobionate, erythromycin propionate, erythromycin stearate, ethambutol hydrochloride, ethionamide, fleroxacin, floxacillin, fludalanine, flumequine, fosfomycin, fosfomycin tromethamine, fumoxicillin, furazolium chloride, furazolium tartrate, fusidate sodium, fusidic acid, gentamicin sulfate, gloximonam, gramicidin, haloprogin, hetacillin, hetacillin potassium, hexedine, ibafloxacin, imipenem, isoconazole, isepamicin, isoniazid, josamycin, kanamycin sulfate, kitasamycin, levofuraltadone, levopropylcillin potassium, lexithromycin, lincomycin, lincomycin hydrochloride, lomefloxacin, lomefloxacin hydrochloride, lomefloxacin mesylate, loracarbef, mafenide, meclocycline, meclocycline sulfosalicylate, megalomicin potassium phosphate, mequidox, meropenem, methacycline, methacycline hydrochloride, methenamine, methenamine hippurate, methenamine mandelate, methicillin sodium, metioprim, metronidazole hydrochloride, metronidazole phosphate, mezlocillin, mezlocillin sodium, minocycline, minocycline hydrochloride, mirincamycin hydrochloride, monensin, monensin sodiumr, nafcillin sodium, nalidixate sodium, nalidixic acid, natainycin, nebramycin, neomycin palmitate, neomycin sulfate, neomycin undecylenate, netilmicin sulfate, neutramycin, nifuradene, nifuraldezone, nifuratel, nifuratrone, nifurdazil, nifurimide, nifiupirinol, nifurquinazol, nifurthiazole, nitrocycline, nitrofurantoin, nitromide, norfloxacin, novobiocin sodium, ofloxacin, onnetoprim, oxacillin sodium, oximonam, oximonam sodium, oxolinic acid, oxytetracycline, oxytetracycline calcium, oxytetracycline hydrochloride, paldimycin, parachlorophenol, paulomycin, pefloxacin, pefloxacin mesylate, penamecillin, penicillin G benzathine, penicillin G potassium, penicillin G procaine, penicillin G sodium, penicillin V, penicillin V benzathine, penicillin V hydrabamine, penicillin V potassium, pentizidone sodium, phenyl aminosalicylate, piperacillin sodium, pirbenicillin sodium, piridicillin sodium, pirlimycin hydrochloride, pivampicillin hydrochloride, pivampicillin pamoate, pivampicillin probenate, polymyxin B sulfate, porfiromycin, propikacin, pyrazinamide, pyrithione zinc, quindecamine acetate, quinupristin, racephenicol, ramoplanin, ranimycin, relomycin, repromicin, rifabutin, rifametane, rifamexil, rifamide, rifampin, rifapentine, rifaximin, rolitetracycline, rolitetracycline nitrate, rosaramicin, rosaramicin butyrate, rosaramicin propionate, rosaramicin sodium phosphate, rosaramicin stearate, rosoxacin, roxarsone, roxithromycin, sancycline, sanfetrinem sodium, sarmoxicillin, sarpicillin, scopafungin, silver ion, sisomicin, sisomicin sulfate, sparfloxacin, spectinomycin hydrochloride, spiramycin, stallimycin hydrochloride, steffimycin, streptomycin sulfate, streptonicozid, sulfabenz, sulfabenzamide, sulfacetamide, sulfacetamide sodium, sulfacytine, sulfadiazine, sulfadiazine sodium, sulfadoxine, sulfalene, sulfamerazine, sulfameter, sulfamethazine, sulfamethizole, sulfamethoxazole, sulfamonomethoxine, sulfamoxole, sulfanitran, sulfasalazine, sulfasomizole, sulfathiazole, sulfazamet, sulfisoxazole, sulfisoxazole acetyl, sulfisboxazole diolamine, sulfomyxin, sulopenem, sultamricillin, suncillin sodium, talampicillin hydrochloride, teicoplanin, temafloxacin hydrochloride, temocillin, tetracycline, tetracycline hydrochloride, tetracycline phosphate complex, tetroxoprim, thiamphenicol, thiphencillin potassium, ticarcillin cresyl sodium, ticarcillin disodium, ticarcillin monosodium, ticlatone, tiodonium chloride, tobramycin, tobramycin sulfate, tosufloxacin, trimethoprim, trimethoprim sulfate, trisulfapyrimidines, troleandomycin, trospectomycin sulfate, tyrothricin, vancomycin, vancomycin hydrochloride, virginiamycin, zorbamycin, penicillin G, tazobactum, ceftrioxone, itraconazole, promethazine, diundecyldimethlammonium, hexadecylpyridinium, benzalkonium, benzethonium, ranitidine, mepenzolate, didecyldimethylammonium, hexadecylpyridinium, streptomycin, isoniazide, miconazole, lidocaine, (2-acetoxyethyl)dodecyloxymethyldimethylammonium, (2-acetoxyethyl)heptyloxymethyldimethylammonium, (2-hydroxyethyl)cyclododecyloxymethyldimethylammonium, or (2-hydroxyethyl)dimethylundecyloxymethylammonium or a cationic derivative thereof.

Examples of antiviral agents that are envisioned as ionic components of embodiment ionic liquid compositions include, but are not limited to acemannan, acyclovir, acyclovir sodium, adefovir, alovudine, alvircept sudotox, amantadine hydrochloride, aranotin, arildone, atevirdine mesylate, pyridine, cidofovir, cipamfylline, cytarabine hydrochloride, delavirdine mesylate, desciclovir, didanosine, disoxaril, edoxudine, enviradene, enviroxime, famciclovir, famotine hydrochloride, fiacitabine, fialuridine, fosarilate, foscarnet sodium, fosfonet sodium, ganciclovir, ganciclovir sodium, idoxuridine, kethoxal, lamivudine, lobucavir, memotine hydrochloride, methisazone, nevirapine, penciclovir, pirodavir, ribavirin, rimantadine hydrochloride, saquinavir mesylate, somantadine hydrochloride, sorivudine, statolon, stavudine, tilorone hydrochloride, trifluridine, valacyclovir hydrochloride, vidarabine, vidarabine phosphate, vidarabine sodium phosphate, viroxime, zalcitabine, zidovudine, zinviroxime, or a cationic derivative thereof.

Examples of pharmaceutically-acceptable carriers include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the disclosed compounds, which matrices are in the form of shaped articles, e.g., films, liposomes, microparticles, or microcapsules. It will be apparent to those persons skilled in the art that certain carriers can be more preferable depending upon, for instance, the route of administration and concentration of composition being administered. Other compounds can be administered according to standard procedures used by those skilled in the art.

Pharmaceutical formulations can include additional carriers, as well as thickeners, diluents, buffers, preservatives, surface active agents and the like in addition to the compounds disclosed herein. Pharmaceutical formulations can also include one or more additional active ingredients such as antimicrobial agents, anti-inflammatory agents, anesthetics, and the like.

The pharmaceutical formulation can be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed compounds can be administered intravenously, intraperitoneally, intramuscularly, subcutaneously, intracavity, or transdermally as is described more fully elsewhere herein.

### Administration and Delivery

In one aspect, disclosed herein are uses of a delivery device to deliver an ionic liquid composition as disclosed herein to a subject. Further, disclosed are methods for delivering an ionic liquid composition to a subject by administering to the subject any of the nutritional supplements, pharmaceutical formulations, controlled release vehicles, delivery and/or devices.

The compositions described herein can be administered to the subject in a number of ways depending on whether local or systemic treatment is desired, and on the area to be treated. Thus, for example, a composition described herein can be administered as an ophthalmic solution and/or ointment to the surface of the eye. Moreover, a compound or pharmaceutical composition can be administered to a subject vaginally, rectally, intranasally, orally, by inhalation, or parenterally, for example, by intradermal, subcutaneous, intramuscular, intraperitoneal, intrarectal, intraarterial, intralymphatic, intravenous, intrathecal and intratracheal routes. Parenteral administration, if used, is generally characterized by injection. Injectables can be prepared in conventional forms, either as liquid solutions or suspensions, solid forms suitable for solution or suspension in liquid prior to injection, or as emulsions. A more recently revised approach for parenteral administration involves use of a slow release or sustained release system such that a constant dosage is maintained. In one example an ionic liquid composition such as lidocaine docusate is contacted to the skin of a subject to provide an anesthetic effect.

Preparations for parenteral administration include sterile aqueous or non-aqueous solutions, suspensions, and emulsions which can also contain buffers, diluents and other suitable additives. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives, such as antimicrobials, anti-oxidants, chelating agents, and inert gases and the like, can also be present.

Formulations for topical administration can include ointments, lotions, creams, gels, drops, suppositories, sprays, liquids and powders. The disclosed ionic liquid compositions having hydrophobic ions can be particularly useful in such applications because they can adhere to the surface longer when exposed to water or other fluids than would a similar hydrophilic salt. Likewise, ionic liquids comprising disinfectant, herbicide, or pesticide ions and hydrophobic counterions can be expected to resist erosion from rainfall. Conventional pharmaceutical carriers, aqueous, powder or oily bases, thickeners and the like can be necessary or desirable. When applied to skin or mucous tissues (e.g., oral applications) ionic liquid compositions containing pharmaceutical actives formulated with highly hydrophobic anions or cations (as appropriate) can have longer durations of adhesion when exposed to water or other fluids than would similar hydrophilic salts applied in these environments. This would be particularly beneficial for sun screens. It should also be noted that disinfectants, pesticides, or herbicides applied to plant leaves can be less prone to be lost by rain even if it follows application.

Techniques for contacting such surfaces and areas with the disclosed ionic liquid compositions can include, spraying, coating, dipping, immersing, or pouring the composition into or onto the surface or area. The precise technique will depend on such factors as the type and amount of infestation or contamination, the size of the area, the amount of composition needed, preference, cost and the like.

The disclosed ionic liquid compositions can be formulated as part of a controlled release vehicle. For example, microspheres and microcapsules, implants, and the like containing liquid bioactive agents are well known, as are methods for their preparation. As such, these methods can be used with the disclosed ionic liquid compositions to produce controlled release vehicles that can release the disclosed ionic liquid composition with a desired release profile.

Also contemplated are pills prepared from the disclosed ionic liquid compositions that are glasses. For example, a glass ionic liquid composition can be cooled to form a pill. That is, glasses are “cast-able” above their glass transition temperature (T_{g}) into shapes with specific surface areas, allowing predictable release/pharmacokinetic properties. Below T_{g}, these disclosed glass ionic liquid composition can be milled into specific shapes and sizes. Alternatively, the disclosed ionic liquid compositions can be tableted as liquids that upon cooling form glasses. Such a method can allow the homogeneous distribution of a pharmaceutical active into the tablet.

Further, the disclosed ionic liquids can be used as carriers for other active compounds, many of which are disclosed herein. For example, prodrugs, ionic and neutral active molecules can be dissolved in the disclosed ionic liquid compositions.

The disclosed ionic liquids can also be encapsulated in a polymer matrix by methods known in the art.

Also, the disclosed ionic liquid compositions can be dissolved in a suitable solvent or carrier as are disclosed herein. This method can enhance the delivery of one or more active ions in the ionic liquid. Further, as is disclosed herein, this method can create a synergistic effect among the various ions present. While not wishing to be bound by theory, the dissociate coefficient of various ions in an ionic liquid can be different in different solvents. Thus, ions in an ionic liquid can dissociate freely in one solvent and cluster in another. This phenomenon can be utilized to provide formulations of compound that are difficult to deliver (e.g., increase the water solubility of steroids). That is, compounds can be formed into an ionic liquid, as described herein, and then dissolved in a suitable solvent to provide an easily deliverable solution. A synertistic effect can be observed upon administration to a subject, when ions cluster and act together, rather than independently.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures and other reaction ranges and conditions that can be used to optimize the product purity and yield obtained from the described process. Only reasonable and routine experimentation will be required to optimize such process conditions. All chemicals used were of analytical grade, purchased from Sigma-Aldrich (Milwaukee, Wis.), and used without further purification unless otherwise noted.

Melting points of all purchased materials were determined in a capillary tube using a MeI-Temp apparatus (Barnstead/Thermo Scientific). NMR data sets were collected on a 300 MHz Bruker instrument using sample concentrations of 50 mM. Infrared spectra were measured using an Avatar 360 FT-IR ESP (ThermoElectron-Nicolet, Waltham, Mass.) equipped with a Smart DuraSamplIR horizontal attenuated total reflectance (HATR) accessory. Raman spectra were measured using a Thermo-Nicolet 470 FTIR with Nd:YAG laser excitation and HgCdTe detector. Viscosity was measured on 1 mL samples with a Viscolab 3000 viscometer (Cambridge Viscosity, Medford, Mass.). Samples were heated to 90° C., except when otherwise specified, and viscosity was recorded in 2 degree increments between 50 and 90° C. with 8 min equilibration at each temperature. Thermal characterization was carried out using a Q100 DSC and a Q500 TGA (both from TA Instruments, New Castle, Del.). DSC was performed using a 5-10 mg sample in a 40 μL aluminum crucible sealed in the glovebox. A temperature range from −60 to 150° C. was scanned twice, at ramp rates of 5° C./min and 10° C./min. TGA was performed under N₂ atmosphere over a temperature range from 25-800° C. with a ramp rate of 20° C. per minute and a sample size of 10 mg.

In general, syntheses of chlorometallate ionic liquids were accomplished by adding 2 molar equivalents of anhydrous ZnCl₂ to 1 molar equivalent of organic chloride salt in a 20 mL vial and flushing three times with argon while stirring with a magnetic stir bar. The stirred mixture was heated to between 60 and 120° C. until a melt formed and all solid was consumed. Molar ratios of 3:1, 1:1, and 1:2 ZnCl₂:organic salt were investigated for ethambutol-HCl, benzethonium-Cl, and ciprofloxacin-HCl and benzethonium-Cl. Benzethonium-Cl was further investigated with 9, 1.5, and 0.67 equivalents ZnCl₂.

The liquid and solid phase presence of multiple chlorozincate species as driving forces for the amorphous form of these ionic liquids was confirmed in each example by Raman spectroscopy. Table 1 shows peaks (in wavenumbers) assigned to four species of chlorozincates over melts formed from 1 molar equivalent of benzethonium chloride and 0.5, 0.67, 1, 1.5, 2.0, or 9.0 molar equivalents of ZnCl₂.

TABLE 1

| | | 0.67 eq. | 1.0 eq. | 1.5 eq. | 2.0 eq. | |
|---|---|---|---|---|---|---|
| | 0.5 eq. | of | of | of | of | 9.0 eq. of |
| Speciation | ZnCl₂ | ZnCl₂ | ZnCl₂ | ZnCl₂ | ZnCl₂ | ZnCl₂ |
| [Zn₄Cl₁₀]²⁻ | 231 | | 235 | 233 | 229 | 229 |
| [Zn₄Cl₄]²⁻ | 278 | 278 | 276 | 274 | | |
| [Zn₃Cl₈]²⁻ | | | | 288 | 290 | |
| [Zn₂Cl₆]²⁻ | | | 310 | 309 | | |
| [Zn₃Cl₈]²⁻ | | | | 340 | 340 | |
| [Zn₄Cl₁₀]²⁻ | | | | | 348 | |

The gas phase presence of multiple chlorozincate species as driving forces for the amorphous form of these ionic liquids was confirmed for benzethonium-Cl—(ZnCl₂)₂ by LC-MS-TOF. Analyses were made using an Agilent 1100 series HPLC coupled to an Agilent 6210 series time-of-flight mass spectrometer via an electrospray ionization source, used in both positive and negative modes. The column was a Higgins Analytical Phalanx C18 reverse phase column and analysis was done using a 5 mM ammonium formate mobile phase and a gradient of 50-90% methanol over 7 minutes. Peaks showing zinc chloride speciation appeared in the negative mode spectra of the IL, but not in the negative mode spectra of the benzethonium chloride starting material. Peaks found for the IL were m/z=168.82, m/z=306.67, and m/z=440.53, which match calculated masses for ZnCl₃⁻ (168.84), Zn₂Cl₅⁻ (306.70), and Zn₃Cl₇⁻ (440.56), respectively.

### Example 1

### Preparation of Phenoxybenzamine-HCl—(ZnCl₂)₂

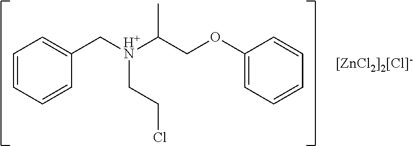

Zinc(II) chloride (273 mg, 2 mmol) was added to a scintillation vial that had been heated, then cooled under vacuum and charged with dry argon. Phenoxybenzamine hydrochloride (340.3 mg, 1 mmol) was added to the vial with a stir bar and the mixture was degassed with stirring at 60° C. The vial was filled with argon, sealed with a cap, and stirred for 24 h at 80° C. to produce a clear, colorless glass. Characterization: T_{g} (glass transition temperature)=30.31° C., T_{dec} (decomposition temperature)=259.0° C., ΔT_{dec} (increase in decomposition temperature relative to HCl starting material)=+45.1° C. NMR spectra for ¹H and ¹³C were obtained in d₆-acetone. The ¹H spectrum showed increased splitting with increasing ZnCl₂ due to formation of enantiomers. Spectra of phenoxybenzamine-HCl were performed with 0, 0.25, 0.5, 1.0, 1.5, 2.0, and 5.0 molar equivalents of ZnCl₂ to observe this effect. Thermal decomposition after a 3 h incubation of the IL at a constant 120° C. was 2.7% of the starting mass. In contrast, the phenoxybenzamine hydrochloride starting material loses 38% of its mass after a similar incubation. The IL was soluble (>1 mM) in acetone, water and phosphate buffered to pH 6.0. The IL was insoluble (<1 mM) in toluene and tetrahydrofuran. Samples were stored under Ar and observed over a period of 6 months during which time no crystalline domains appeared. Model-free isoconversional kinetic analysis (for more detail, see: Doyle, C. D., J. Appl. Polym. Sci. 1962, 6:639-642; and Long, G. T. et al., J. Pharm. Sci. 2002, 91:800-809) of TGA data was used to estimate increases in shelf life due to IL formulation for phenoxybenzamine hydrochloride and phenoxybenzamine-HCl—(ZnCl₂)₂. The onset of mass loss indicates loss of HCl during cyclization of the 2-chloroethylamine group, the most common decomposition pathway for phenoxybenzamine hydrochloride (for more detail, see: Adams, W. P. et al., Int. J. Pharm. 1985, 25:293-312). Formulation as an ionic liquid improves the shelf life (time to reach 5% decomposition) of solid phenoxybenzamine hydrochloride at 20° C. by 7.7-fold to 15.8 years. Compared with the 4-day refrigerator shelf life of a standard formulation of phenoxybenamine-HCl in syrup and propylene glycol, (described in: Glass, B. D. et al., J Pharm Pharm Sci 2006, 9:398-426) the IL showed a 1500-fold improvement. The manufacturer's shelf life for 10 mg phenoxybenzamine hydrochloride tablets is 2 years, or 730 days, and the calculated value for solid phenoxybenzamine hydrochloride (5% decay at 20° C.) was 750 days (2.05 years), indicating good agreement of the calculation with actual shelf life. The most common decomposition pathway for phenoxybenzamine hydrochloride is loss of hydrochloride during cyclization of the 2-chloroethylamine group (for more detail, see: Adams, W. P. et al., Int. J. Pharm. 1985, 25:293-312) and we expect this is the earliest mass loss observed by TGA. The IL phenoxybenzamine-HCl—(ZnCl₂)₂ is a stabilized, amorphous formulation with shelf life greater than that of the most common crystalline form.

### Example 2

### Preparation of Homatropine-HCl—(ZnCl₂)₂

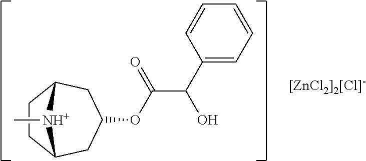

Zinc(II) chloride (273 mg, 2 mmol) was added to a scintillation vial that had been heated, then cooled under vacuum and charged with dry argon. Homatropine hydrochloride (311.8 mg, 1 mmol) was added to the vial with a stir bar and the mixture was degassed with stirring at 60° C. The vial was filled with argon, sealed with a cap, and stirred for 24 h at 120° C. to produce a clear, colorless glass. Characterization: T_{g}=62.18° C., T_{dec}=274.5° C., ΔT_{dec}=+7.8° C. The IL was soluble (>1 mM) in dimethylsulfoxide and insoluble (<1 mM) in toluene. Samples were stored under Ar and observed over a period of 6 months during which time no crystalline domains appeared.

### Example 3

### Preparation of Benzethonium-Cl—(ZnCl₂)₂

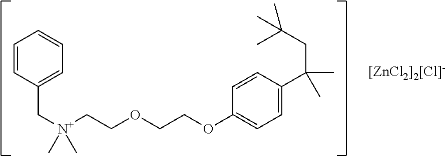

Zinc(II) chloride (273 mg, 2 mmol) was added to a scintillation vial that had been heated, then cooled under vacuum and charged with dry argon. Benzethonium chloride (448.1 mg, 1 mmol) was added to the vial with a stir bar and the mixture was degassed with stirring at 60° C. The vial was filled with argon, sealed with a cap, and stirred for 48 h at 120° C. to produce a clear, colorless viscous liquid. Molar ratios of 1 equivalent ZnCl₂ to 1 equivalent benzethonium chloride, as well as 3/1, 1/2, 9/1, 3/2 and 2/3 molar ratios of ZnCl₂/benzethonium chloride were also tested, but were more viscous than the described 2/1 example. Characterization: T_{g}=8.58° C., T_{dec}=291.8° C., ΔT_{dec}=+110.3° C. Viscosity at 90° C. was 8700 cP (centipoise). Thermal decomposition after a 3 h incubation of the IL at a constant 160° C. was 0.9% of the starting mass. In contrast, the benzethonium chloride starting material loses 33% of its mass after a similar incubation. The IL was soluble (>1 mM) in chloroform and acetone. The IL was insoluble (<1 mM) in water, decanoic acid, squalene, light mineral oil, Tween 80, Tween 20, and Triton-X 100. Minimum inhibitory concentrations (MICs) were determined by the broth microdilution method using a panel of gram-negative and gram-positive bacteria and the results are shown in Table 2. Briefly, benzethonium-Cl or benzethonium-Cl—(ZnCl₂)₂ was diluted into LB medium, followed by inoculation of one of a panel of five cultured gram-negative bacteria and two gram-positive bacteria, specifically Escherichia coli K12, Salmonella typhimurium LT2, Staphylococcus epidermis (clinical isolate from blood), Burkholderia thailandensis E264, Pseudomonas aeruginosa (clinical isolate from sputum), Bacillus anthracis Sterne, and Bacillus thuringiensis HD31. Bacteria were streaked from glycerol-frozen stocks onto Luria Bertani agar plates and incubated for 1 d at 37° C. Cells from the plate were inoculated into LB media and incubated for 12 h at 37° C. with shaking (200 rpm). The bacteria were diluted in LB to 10⁵ CFU and were subsequently added to 100 μL of LB broth containing various concentrations of benzethonium-Cl or benzethonium-Cl—(ZnCl₂)₂ in a 96-well microtiter plate. The final concentrations tested were 0, 0.5, 1, 2, 4, 8, 16, 32, 64, 128, and 256 μg/mL. The plates were incubated for 24 h at 37° C. The MIC in milligrams per milliliter (mg/mL) was defined as the lowest of these concentrations that did not support observable bacterial growth after incubation. Samples were stored under Ar and observed over a period of 6 months during which time no crystalline domains appeared.

TABLE 2

| | | | Fold | Fold |
|---|---|---|---|---|
| | benze- | Benze- | improve- | improve- |
| | thonium- | thonium- | ment, | ment, |
| Species | Cl—(ZnCl2)2 | Cl | mass ratio | molar ratio |
| Gram-negative | | | | |
| Escherichia | 16 | 16 | same | 1.6x |
| coli K12 | | | | |
| Salmonella | 64 | 128 | 2x | 3.2x |
| typhimurium | | | | |
| LT2 | | | | |
| Staphylococcus | 8 | 16 | 2x | 3.2x |
| epidermis | | | | |
| (clinical isolate) | | | | |
| Burkholderia | 64 | >256 | >4x | >6.4x |
| thailandensis | | | | |
| E264 | | | | |
| Pseudomonas | 2 | 16 | 8x | 12.9x |
| aeruginosa | | | | |
| (clinical isolate) | | | | |
| Gram-Positive | | | | |
| Bacillus | 2 | 2 | same | 1.6x |
| anthracis | | | | |
| Sterne | | | | |
| Bacillus | 2 | 2 | same | 1.6x |
| thuringiensis | | | | |
| HD31 | | | | |

### Example 4

### Preparation of Lysine-HCl—(ZnCl₂)₂

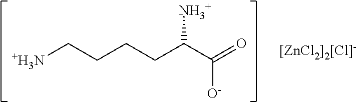

Zinc(II) chloride (273 mg, 2 mmol) was added to a scintillation vial that had been heated, then cooled under vacuum and charged with dry argon. L-Lysine hydrochloride (182.7 mg, 1 mmol) was added to the vial with a stir bar and the mixture was degassed with stirring at 60° C. The vial was filled with argon, sealed with a cap, and stirred for 48 h at 120° C. to produce a clear, colorless glass. Characterization: T_{g}=−22.57° C., T_{dec}=332.8° C., ΔT_{dec}=+55.5° C. Viscosity was 24240 cP at 145° C. The IL was soluble (>1 mM) in acetone. Samples were stored under Ar and observed over a period of 6 months during which time no crystalline domains appeared.

### Example 5

### Preparation of Ranitidine-HCl—(ZnCl₂)₂

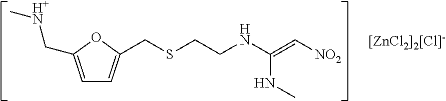

Zinc(II) chloride (273 mg, 2 mmol) was added to a scintillation vial that had been heated, then cooled under vacuum and charged with dry argon. Ranitidine hydrochloride (350.9 mg, 1 mmol) was added to the vial with a stir bar and the mixture was degassed with stirring at 60° C. The vial was filled with argon, sealed with a cap, and stirred for 5 h at 70° C. to produce a dark yellow glass. Characterization: T_{g}=−21.89° C., T_{dec}=230.9° C., ΔT_{dec}=+14.1° C. The IL was soluble (>1 mM) in acetone. Samples were stored under Ar and observed over a period of 6 months during which time no crystalline domains appeared.

### Example 6

### Preparation of Procainamide-HCl—(ZnCl₂)₂

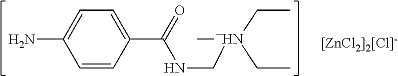

Zinc(II) chloride (273 mg, 2 mmol) was added to a scintillation vial that had been heated, then cooled under vacuum and charged with dry argon. Procainamide hydrochloride (271.8 mg, 1 mmol) was added to the vial with a stir bar and the mixture was degassed with stirring at 60° C. The vial was filled with argon, sealed with a cap, and stirred for 4 h at 60° C. and then for 4 h at 120° C. to produce a clear, colorless glass. Characterization: T_{g}=−16.99° C., T_{dec}=291.0° C., ΔT_{dec}=+11.3° C. The IL was soluble (>1 mM) in acetone. The IL was insoluble (<1 mM) in dimethylsulfoxide, water, and toluene). Samples were stored under Ar and observed over a period of 6 months during which time no crystalline domains appeared.

### Example 7

### Preparation of Ethambutol-(HCl)₂—(ZnCl₂)₂

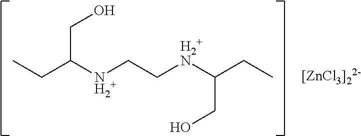

Zinc(II) chloride (273 mg, 2 mmol) was added to a scintillation vial that had been heated, then cooled under vacuum and charged with dry argon. Ethambutol dihydrochloride (277.2 mg, 1 mmol) was added to the vial with a stir bar and the mixture was degassed with stirring at 60° C. The vial was filled with argon, sealed with a cap, and stirred for 48 h at 120° C. to produce a clear, colorless, highly viscous liquid. Characterization: T_{g}=13.74° C., T_{dec}=216.5° C., ΔT_{dec}=−33.6° C. Viscosity at 90° C. was 17350 cP. The IL was soluble in acetone at 1 mM concentration. Samples were stored under Ar and observed over a period of 6 months during which time no crystalline domains appeared. Molar ratios of 1 equivalent ZnCl₂ to 1 equivalent ethambuthol dihydrochloride, as well as 3/1 and 1/2 ZnCl₂/ethambutol dihydrochloride were also tested, but were more viscous than the described 2/1 example.

### Example 8

### Preparation of Nicardipine-HCl—(ZnCl₂)₂

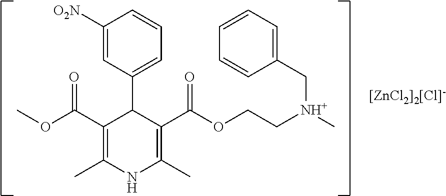

Zinc(II) chloride (273 mg, 2 mmol) was added to a scintillation vial that had been heated, then cooled under vacuum and charged with dry argon. Nicardipine hydrochloride (516.0 mg, 1 mmol) was added to the vial with a stir bar and the mixture was degassed with stirring at 60° C. The vial was filled with argon, sealed with a cap, and stirred for 8 h at 90° C. to produce a clear, yellow glass. Characterization: T_{g}=39.2° C., T_{dec}=302.2° C., ΔT_{dec}=+75.5° C. The IL is soluble (≧50 mM) in acetone and dimethylsulfoxide and insoluble (<1 mM) in toluene. Samples were stored under Ar and observed over a period of 6 months during which time no crystalline domains appeared.

### Example 9

### Preparation of Imipramine-HCl—(ZnCl₂)₂

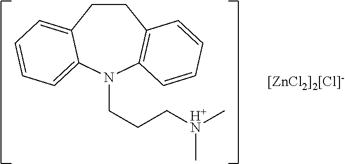

Zinc(II) chloride (273 mg, 2 mmol) was added to a scintillation vial that had been heated, then cooled under vacuum and charged with dry argon. Imipramine hydrochloride (316.9 mg, 1 mmol) was added to the vial with a stir bar and the mixture was degassed with stirring at 60° C. The vial was filled with argon, sealed with a cap, and stirred for 24 h at 100° C. to produce a clear, colorless glass. Characterization: T_{g}=24.6° C., T_{dec}=267.2° C., ΔT_{dec}=+21.4° C. Thermal decomposition after a 3 h incubation of the IL at a constant 200° C. was 12% of the starting mass. In contrast, the benzethonium chloride starting material loses 49% of its mass after a similar incubation. The IL is soluble (>50 mM) in acetone and acetonitrile. Samples were stored under Ar and observed over a period of 6 months during which time no crystalline domains appeared.

### Example 10

### Preparation of Benzethonium-Cl—(CoCl₂)₂

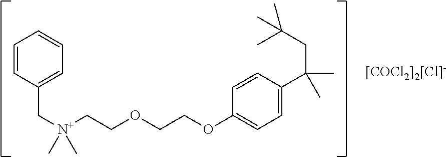

Cobalt(II) chloride (259.6 mg, 2 mmol) was added to a scintillation vial that had been heated, then cooled under vacuum and charged with dry argon. Benzethonium chloride (448.1 mg, 1 mmol) was added to the vial with a stir bar and the mixture was degassed with stirring at 60° C. The vial was filled with argon, sealed with a cap, and stirred for 24 h at 120° C. to produce a purple viscous liquid. Characterization: T_{g}=15.0° C. Samples were stored under Ar and observed over a period of 6 months during which time no crystalline domains appeared.

### Example 11

### Preparation of Benzethonium-Cl—(FeCl₃)₂

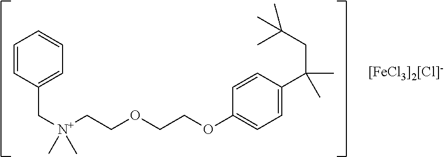

Iron(II) chloride (324.4 mg, 2 mmol) was added to a scintillation vial that had been heated, then cooled under vacuum and charged with dry argon. Benzethonium chloride (448.1 mg, 1 mmol) was added to the vial with a stir bar and the mixture was degassed with stirring at 60° C. The vial was filled with argon, sealed with a cap, and stirred for 24 h at 120° C. to produce a purple viscous liquid. Characterization: T_{g}=−17.1° C. Samples were stored under Ar and observed over a period of 6 months during which time no crystalline domains appeared.

### Example 12

### Preparation of Benzethonium-Cl—(SnCl₂)₂

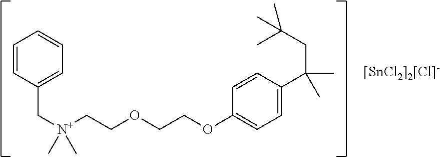

Tin(II) chloride (379.2 mg, 2 mmol) was added to a scintillation vial that had been heated, then cooled under vacuum and charged with dry argon. Benzethonium chloride (448.1 mg, 1 mmol) was added to the vial with a stir bar and the mixture was degassed with stirring at 60° C. The vial was filled with argon, sealed with a cap, and stirred for 24 h at 120° C. to produce a white viscous liquid. Characterization: T_{g}=5.8° C. Samples were stored under Ar and observed over a period of 6 months during which time no crystalline domains appeared.

Zinc-based IL forms of pharmaceuticals are generally more water-soluble than the chloride salt starting materials. Improved water solubility would enable ILs to dissolve more quickly in the stomach, increasing efficacy and speeding the drug's action. Solubility of phenoxybenzamine hydrochloride and phenoxybenzamine-HCl—(ZnCl₂)₂ at 1 mM in unbuffered water (pH 6.5) was evaluated by eye and by UV-Vis spectroscopy after filtration through a 0.45 μm syringe filter. The phenoxybenzamine-HCl—(ZnCl₂)₂ ionic liquid appeared to completely dissolve at 1 mM (0.34 mg/mL), and A₂₆₈ of the filtrate (A₂₆₈=1.56) was 5× that of the phenoxybenzamine hydrochloride solution (A₂₆₈=0.30), indicating that formulation of phenoxybenzamine hydrochloride as the amorphous ZnCl₂-based IL increases the water solubility of the pharmaceutical.

Another example of improved solubility upon formulation as described herein is shown for ranitidine-HCl—(ZnCl₂)₂. Upon introduction of 1 mM IL into a stirred cuvette of water at pH 6.5 and monitoring by UV-Vis absorption at 314 nm, maximal absorption is reached after 30 seconds of stirring, while maximal absorption for a 1 mM solution of ranitidine-HCl in water at pH 6.5 is not reached until after 10 minutes of stirring.

An example of the utility of these compositions as antifouling and antimicrobial surfaces has been shown against biofilms of Pseudomonas aeruginosa and Escherichia coli. The antimicrobial efficacy of described compositions has been shown in two formats, namely a) layering neat IL over established biofilm growths and b) coating culture plates with IL and observing the biofilm growth on the coated surface. The assay for the first format was performed as follows and as established in literature (see: Leid, J. G. et al., J Immunol 2005, 175:7512-7518). P. aeruginosa (clinical isolate from sputum) was isolated from a single colony on LB agar solid medium and grown to confluence for 12-15 h at 37° C. with shaking (200 rpm). Following this incubation, the culture was diluted 1:100 in fresh LB media and incubated at 37° C. with shaking for 4 h, at which time it was further diluted 1:50 into fresh LB media. From this dilution, 100 μL of culture was used to inoculate each wells of a 96-well PVC microtiter plate. The plate was incubated at 37° C. for a total of 72 h. The LB media was decanted and replaced with an equal volume of fresh LB media every 24 h post inoculation. After the 72 h growth period, the LB was decanted from the biofilms. Each test compound (LB, light mineral oil, 1-butyl-1-methypyrrolidinium bistriflimide (BMP-NTf₂), ZnCl₂, benzethonium-Cl, NaCl, or 1:1 benzethonium-Cl—(ZnCl₂)₂:1-butyl-1-methylpyrrolidinium bistriflimide) was added and the biofilms were challenged for 6 h at 37° C. The ionic liquids and light mineral oil were added neat. The concentrations of ZnCl₂, benzethonium-Cl, and NaCl were 1.8 M, 78 mM, and 5 M, respectively, all in LB. Following the challenge period, the test compounds were removed, the biofilms washed gently (without agitation) with 100 μL of LB per well. The wash media was removed and replaced with fresh LB (100 μL per well). Biofilms were then gently sonicated using a platform sonicator (Gilson) and remaining bacteria were enumerated by serially diluting and plating on appropriate agar media. Colonies were counted and recorded as colony forming units (CFU). Neat 1:1 benzethonium-Cl—(ZnCl₂)₂:BMP-NTf₂ reduced the attached cell count of Pseudomonas aeruginosa biofilms (72 h biofilm age) by four orders of magnitude (1×10⁴ cfu/mL viable bacteria, p<0.01) from that of untreated control, or cultures treated with mineral oil, a control for viscosity. The strain is a clinical isolate from sputum. The six-hour exposure used in these studies is significantly shorter than the days, weeks, or months usually required for antibiotic treatment of biofilms in the clinic. The neat IL reduced the cell count by two orders of magnitude more than 5 M NaCl, a control for ionic strength (1×10⁶ cells, p<0.05). The values for neat BMP-NTf₂ and 1.8 M ZnCl₂ were 2×10⁵ (p<0.05) and 4×10⁵ (p<0.01), respectively. In growth medium, 78 mM benzethonium chloride formed a viscous, non-dispersible precipitate, which impeded quantification of benzethonium chloride efficacy with statistical certainty. We conservatively estimate the viable dispersed cell count in some exposures to be 5×10⁴ cfu/mL. A smaller amount of precipitate that contains no viable bacteria is seen in exposures with the IL. The antimicrobial action associated with the 1:1 benzethonium-Cl—(ZnCl₂)₂:BMP-NTf₂ ionic liquid is a promising starting point for development of an antibiofilm treatment.

A second example of clear, colorless antimicrobial films formed by compositions described herein was shown with an assay that involved coating culture plates with IL and observing the biofilm growth on the coated surface. Standard 96-well polystyrene culture plates were covered with 100 μL of the benzethonium-Cl—(ZnCl₂)₂ ionic liquid per well. Aliquots of 100 μL of either a Staphylococcus aureus culture, an E. coli culture, or a Pseudomonas aeruginosa culture were added to each coated well, the plate was fed with fresh media every 24 h, and cells were counted after 72 h, as described above. The number of cells in the treated wells was below the limit of detection of the assay of 1000 cfu/mL, as compared with normal cell counts for the LB control wells, indicating high antimicrobial efficacy of the IL coating. Because bacteria colonize a surface prior to barnacles and other organisms, proof of antibacterial effects is key to showing anti-biofouling potential in aquatic environments.

Other advantages which are obvious and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

What is claimed is:

1. An amorphous formulation of a pharmaceutical, comprising: a composition of the formula [A]ₓ[MₚCl_{q}]_{y}, said composition being an ionic liquid with a melting temperature below 150° C., wherein [MₚCl_{q}] is a metal chloride, x is 1, 2, 3, or 4, p is 1, 2, 3, or 4, q is 1, 2, 3, 4, 5, 6, 7, 8, or 9, and y is 1 or 2, wherein each A is a cation that is a pharmaceutical or a cation precursor that is a pharmaceutical.

2. The composition of claim 1, wherein [MₚCl_{q}] comprises Zn₄Cl₁₀, ZnCl₄, Zn₃Cl₈, Zn₂Cl₆, ZnCl₃, Zn₂Cl₅, Zn₃Cl₇, or combinations thereof.

3. The composition of claim 1, wherein the composition further comprises a solvent, preservative, dye, colorant, thickener, surfactant, a viscosity modifier, or a mixture thereof at less than about 10 wt. % of the total ionic liquid composition.

4. The composition of claim 1, wherein the composition further comprises a nonionic pharmaceutical active, nutraceutical, food additive, or mixture thereof.

5. The composition of claim 1, wherein A is pharmaceutical active.

6. The composition of claim 1, wherein the cation or cation precursor comprises benzethonium, ranitidinium, phenoxybenzamine, homatropine, lysine, procainamide, ethambutol, nicardipine, imipramine or a cationic derivative thereof.

7. The composition of claim 4, wherein the pharmaceutical active comprises aspirin, chlordiazepoxide HCl, isoniazid, penicillin, sulfamidochrysoidine, cisplatin, 6-mercaptopurine, chimeric anti-CD20 antibody, paclitaxel, phenobarbital, fluoxetine, fexofenadine, rofecoxib, quinine, ivermectin, L-dopa, 10-(3-dimethylaminoproply)-2-chlorphenothiazine, salvarsan, cimetidine, zidovudine, indinavir sulfate, salbutamol, digoxin, fluride, zidovudine, erythropoietin, hydrocortisone, insulin, oral contraceptive, oxytocin, mifepristone, thyroxine, thalidomide, cyclosporine, fentanyl, methadone, morphine, botox, vitamins, methylphenidate, sildenafil, pantoprazole, rabeprazole, risedronate, alendronate, losartan, fosinopril, atorvastatin, pravastatin, montelukast, or a cationic derivative thereof.

8. The composition of claim 1, wherein A comprises the cation of 2-aminothiazole, 6-azauridine, 6-mercaptopurine, 9-aminocamptothecin, acetarsone, acetazolamide, acetosulfone, acetylcysteine, acetylpheneturide, acranil, acrivastine, adrenalone, albutoin, aldioxa, alibendol, alizapride, alkofanone, allopurinol, ambenonium, ambroxol, ambutonium, amexinium, aminitrozole, amiphenazole, amisulpride, anagrelide, arsanilate, arshpenamine, asoxime, azidamfenicol, benfluorex, benfunolol, benserazide, benzaepril, benzilonium, benznidazole, benzoxonium, benzoylpas, berberine, bibenzonium, brequinar, brodimoprim, bromhexine, bromopride, bromo-salicylchloranilide, bucillamine, buformin, bufuralol, bumadizon, bunitrolol, buthalital, cabergoline, carbachol, carbarsone, carbidopa, cariporide, carnitine, caroxazone, celecoxib, cephaeline, cetalkonium, chloguanide, chloramphenicol, chlordantoin, chlorisonclamine, chloroquine, cholic acid, choline, ciclonicate, cilostazol, cimetidine, cinepazide, cinitapride, cisapride, clanobutin, clometocillin, clomiphene, cloricromen, clortermine, cyamelide, cyclazocine, dapsone, dimefline, diphetarsone, distigmine, dithiazanine, donepezil, dopamine, echothiophate, edrophonium, efavirenz, elformithine, enfenamic acid, etifoxine, etodroxizine, fampridine, fazadinium, fendiline, fenproporex, finasteride, flufenamic acid, flumethiazide, fluoxetine, flurazepam, flutamide, fomepizole, fominoben, hexafluorenium, hydroxystilbamidine, ibudilast, indalpine, indeloxazine, ipratropium, irsogladine, isobutyl p-aminobenzoate, isoetharine, levosimendan, lisuride, lodoxamide, mesalamine, mesoxalate, metformin, methionine, methoxyphenamine, methyl hexaneamine, methylene blue, methylthiouracil, metrons, metyrosine, minoxidil, nadoxolol, nafronyl, naphazoline, naratriptan, neostigmine, nicametate, niclosamide, nicorandil, nilutamide, niridazole, nitazoxanide, nordefrin, obidoxime, omapatrilat, orotic acid, oxaceprol, oxitropium, ozagrel, pamidronic acid, p-aminobenzoic acid, p-aminopropiophenon, pelletierine, pemoline, penicillamine, pentamethonium, pentamidine, pentoxifylline, pergolid, perhexiline, phenmetrazine, phentolamine, phenylephrine, picotamide, pidotimod, pimobendan, pinacidil, pramipexole, prenylamine, prinomastat, pyridinol carbamate, pyridoxine, pyrimethamine, ramatroban, remoxipride, repinotan, riluzole, ritodrine, succinimide, succinylcholine, sulfamethoxazole, sulfapyridine, sulpiride, synephrine, tamsulosin, tedisamil, tenuazonic acid, terazosin, terbutaline, tetrazepam, tetroxoprim, thiabendazole, thiamine, thiopental, tiapride, timonacid, tiopronin, tiotropium, tizanidine, tolazoline, tropicamide, valnoctamide, zafirlukast, or a cationic derivative thereof.

9. The composition of claim 4, wherein the pharmaceutical active comprises an antibacterial.

10. The composition of claim 9, wherein the antibacterial comprises acedapsone, fosmidomycin, acetosulfone sodium, alamecin, alexidine, amdinocillin, amdinocillin pivoxil, amicycline, amifloxacin, amifloxacin mesylate, amikacin, amikacin sulfate, aminosalicylic acid, aminosalicylate sodium, amoxicillin, amphomycin, ampicillin, ampicillin sodium, apalcillin sodium, apramycin, aspartocin, astromicin sulfate, avilamycin, avoparcin, azithromycin, azlocillin, azlocillin sodium, bacampicillin hydrochloride, bacitracin, bacitracin methylene disalicylate, bacitracin zinc, bambermycins, benzoylpas calcium, berythromycin, betamicin sulfate, biapenem, biniramycin, biphenamine hydrochloride, bispyrithione magsulfex, butikacin, butirosin sulfate, capreomycin sulfate, carbadox, carbenicillin disodium, carbenicillin indanyl sodium, carbenicillin phenyl sodium, carbenicillin potassium, carumonam sodium, cefaclor, cefadroxil, cefamandole, cefamandole nafate, cefamandole sodium, cefaparole, cefatrizine, cefazaflur sodium, cefazolin, cefazolin sodium, cefbuperazone, cefdinir, cefepime, cefepime hydrochloride, cefetecol, cefixime, cefmenoxime hydrochloride, cefmetazole, cefmetazole sodium, cefonicid monosodium, cefonicid sodium, cefoperazone sodium, ceforanide, cefotaxime sodium, cefotetan, cefotetan disodium, cefotiam hydrochloride, cefoxitin, cefoxitin sodium, cefpimizole, cefpimizole sodium, cefpiramide, cefpiramide sodium, cefpirome sulfate, cefpodoxime proxetil, cefprozil, cefroxadine, cefsulodin sodium, ceftazidime, ceftibuten, ceftizoxime sodium, ceftriaxone sodium, cefuroxime, cefuroxime axetil, cefuroxime pivoxetil, cefuroxime sodium, cephacetrile sodium, cephalexin, cephalexin hydrochloride, cephaloglycin, cephaloridine, cephalothin sodium, cephapirin sodium, cephradine, cetocycline hydrochloride, cetophenicol, chloramphenicol, chloramphenicol palmitate, chloramphenicol pantothenate complex, chloramphenicol sodium succinate, chlorhexidine phosphanilate, chloroxylenol, chlortetracycline bisulfate, chlortetracycline hydrochloride, cinoxacin, ciprofloxacin, ciprofloxacin hydrochloride, cirolemycin, clarithromycin, clinafloxacin hydrochloride, clindamycin, clindamycin hydrochloride, clindamycin palmitate hydrochloride, clindamycin phosphate, clofazimine, cloxacillin benzathine, cloxacillin sodium, cloxyquin, colistimethate sodium, colistin sulfate, coumermycin, coumermycin sodium, cyclacillin, cycloserine, dalfopristin, dapsone, daptomycin, demeclocycline, demeclocycline hydrochloride, demecycline, denofungin, diaveridine, dicloxacillin, dicloxacillin sodium, dihydrostreptomycin sulfate, dipyrithione, dirithromycin, doxycycline, doxycycline calcium, doxycycline fosfatex, doxycycline hyclate, droxacin sodium, enoxacin, epicillin, epitetracycline hydrochloride, erythromycin, erythromycin acistrate, erythromycin estolate, erythromycin ethylsuccinate, erythromycin gluceptate, erythromycin lactobionate, erythromycin propionate, erythromycin stearate, ethambutol hydrochloride, ethionamide, fleroxacin, floxacillin, fludalanine, flumequine, fosfomycin, fosfomycin tromethamine, fumoxicillin, furazolium chloride, furazolium tartrate, fusidate sodium, fusidic acid, gentamicin sulfate, gloximonam, gramicidin, haloprogin, hetacillin, hetacillin potassium, hexedine, ibafloxacin, imipenem, isoconazole, isepamicin, isoniazid, josamycin, kanamycin sulfate, kitasamycin, levofuraltadone, levopropylcillin potassium, lexithromycin, lincomycin, lincomycin hydrochloride, lomefloxacin, lomefloxacin hydrochloride, lomefloxacin mesylate, loracarbef, mafenide, meclocycline, meclocycline sulfosalicylate, megalomicin potassium phosphate, mequidox, meropenem, methacycline, methacycline hydrochloride, methenamine, methenamine hippurate, methenamine mandelate, methicillin sodium, metioprim, metronidazole hydrochloride, metronidazole phosphate, mezlocillin, mezlocillin sodium, minocycline, minocycline hydrochloride, mirincamycin hydrochloride, monensin, monensin sodiumr, nafcillin sodium, nalidixate sodium, nalidixic acid, natainycin, nebramycin, neomycin palmitate, neomycin sulfate, neomycin undecylenate, netilmicin sulfate, neutramycin, nifuradene, nifuraldezone, nifuratel, nifuratrone, nifurdazil, nifurimide, nifiupirinol, nifurquinazol, nifurthiazole, nitrocycline, nitrofurantoin, nitromide, norfloxacin, novobiocin sodium, ofloxacin, onnetoprim, oxacillin sodium, oximonam, oximonam sodium, oxolinic acid, oxytetracycline, oxytetracycline calcium, oxytetracycline hydrochloride, paldimycin, parachlorophenol, paulomycin, pefloxacin, pefloxacin mesylate, penamecillin, penicillin G benzathine, penicillin G potassium, penicillin G procaine, penicillin G sodium, penicillin V, penicillin V benzathine, penicillin V hydrabamine, penicillin V potassium, pentizidone sodium, phenyl aminosalicylate, piperacillin sodium, pirbenicillin sodium, piridicillin sodium, pirlimycin hydrochloride, pivampicillin hydrochloride, pivampicillin pamoate, pivampicillin probenate, polymyxin B sulfate, porfiromycin, propikacin, pyrazinamide, pyrithione zinc, quindecamine acetate, quinupristin, racephenicol, ramoplanin, ranimycin, relomycin, repromicin, rifabutin, rifametane, rifamexil, rifamide, rifampin, rifapentine, rifaximin, rolitetracycline, rolitetracycline nitrate, rosaramicin, rosaramicin butyrate, rosaramicin propionate, rosaramicin sodium phosphate, rosaramicin stearate, rosoxacin, roxarsone, roxithromycin, sancycline, sanfetrinem sodium, sarmoxicillin, sarpicillin, scopafungin, silver ion, sisomicin, sisomicin sulfate, sparfloxacin, spectinomycin hydrochloride, spiramycin, stallimycin hydrochloride, steffimycin, streptomycin sulfate, streptonicozid, sulfabenz, sulfabenzamide, sulfacetamide, sulfacetamide sodium, sulfacytine, sulfadiazine, sulfadiazine sodium, sulfadoxine, sulfalene, sulfamerazine, sulfameter, sulfamethazine, sulfamethizole, sulfamethoxazole, sulfamonomethoxine, sulfamoxole, sulfanitran, sulfasalazine, sulfasomizole, sulfathiazole, sulfazamet, sulfisoxazole, sulfisoxazole acetyl, sulfisboxazole diolamine, sulfomyxin, sulopenem, sultamricillin, suncillin sodium, talampicillin hydrochloride, teicoplanin, temafloxacin hydrochloride, temocillin, tetracycline, tetracycline hydrochloride, tetracycline phosphate complex, tetroxoprim, thiamphenicol, thiphencillin potassium, ticarcillin cresyl sodium, ticarcillin disodium, ticarcillin monosodium, ticlatone, tiodonium chloride, tobramycin, tobramycin sulfate, tosufloxacin, trimethoprim, trimethoprim sulfate, trisulfapyrimidines, troleandomycin, trospectomycin sulfate, tyrothricin, vancomycin, vancomycin hydrochloride, virginiamycin, zorbamycin, penicillin G, tazobactum, ceftrioxone, itraconazole, promethazine, diundecyldimethlammonium, hexadecylpyridinium, benzalkonium, benzethonium, ranitidine, mepenzolate, didecyldimethylammonium, hexadecylpyridinium, streptomycin, isoniazide, miconazole, lidocaine, (2-acetoxyethyl)dodecyloxymethyldimethylammonium, (2-acetoxyethyl)heptyloxymethyldimethylammonium, (2-hydroxyethyl)cyclododecyloxymethyldimethylammonium, or (2-hydroxyethyl)dimethylundecyloxymethylammonium or a cationic derivative thereof.

10. The composition of claim 4, wherein the pharmaceutical active comprises an antiviral.

11. The composition of claim 10, wherein the antiviral comprises acemannan, acyclovir, acyclovir sodium, adefovir, alovudine, alvircept sudotox, amantadine hydrochloride, aranotin, arildone, atevirdine mesylate, pyridine, cidofovir, cipamfylline, cytarabine hydrochloride, delavirdine mesylate, desciclovir, didanosine, disoxaril, edoxudine, enviradene, enviroxime, famciclovir, famotine hydrochloride, fiacitabine, fialuridine, fosarilate, foscarnet sodium, fosfonet sodium, ganciclovir, ganciclovir sodium, idoxuridine, kethoxal, lamivudine, lobucavir, memotine hydrochloride, methisazone, nevirapine, penciclovir, pirodavir, ribavirin, rimantadine hydrochloride, saquinavir mesylate, somantadine hydrochloride, sorivudine, statolon, stavudine, tilorone hydrochloride, trifluridine, valacyclovir hydrochloride, vidarabine, vidarabine phosphate, vidarabine sodium phosphate, viroxime, zalcitabine, zidovudine, zinviroxime, or a cationic derivative thereof.

12. A method of inhibiting or treating microbial infection in a subject, comprising administering to a subject an effective amount of a composition of the formula [A]ₓ[MₚCl_{q}]_{y}, said composition being an ionic liquid with a melting temperature below 150° C., wherein [MₚCl_{q}] is a metal chloride, x is 1, 2, 3, or 4, p is 1, 2, 3, or 4, q is 1, 2, 3, 4, 5, 6, 7, 8, or 9, and y is 1 or 2, wherein each A is a cation that is a pharmaceutical or a cation precursor that is a pharmaceutical.

13. A method of inhibiting or treating microbial contamination on a surface comprising contacting the surface with an effective amount of the a composition of the formula [A]ₓ[MₚCl_{q}]_{y}, said composition being an ionic liquid with a melting temperature below 150° C., wherein [MₚCl_{q}] is a metal chloride, x is 1, 2, 3, or 4, p is 1, 2, 3, or 4, q is 1, 2, 3, 4, 5, 6, 7, 8, or 9, and y is 1 or 2, wherein each A is a cation that is a pharmaceutical or a cation precursor that is a pharmaceutical.
